(19) United States
(12) Patent Application Publication
(10) Pub. No.: US 2025/0034129 A1
(43) Pub. Date: Jan. 30, 2025

(54) CRYSTALLINE FORMS OF (6S,7S)-6-FLUORO-7-(2-FLUORO-5-METHYLPHENYL)-3-(TETRAHYDRO-2H-PYRAN-4-YL)-5,6,7,8-TETRAHYDROPYRIDO[2,3-d]PYRIMIDINE-2,4(1H,3H)-DIONE

(71) Applicant: MYOKARDIA, INC., Brisbane, CA (US)

(72) Inventors: Ying YU, San Ramon, CA (US); David Aimin ZHANG, Castro Valley, CA (US); Juan WANG, Foster City, CA (US); Neal HUANG, Oakland, CA (US); Jonathan LOUGHREY, Edinburgh, CA (US); Lorraine SHARP, Edinburgh (GB); Catherine DALENS, Sana (FR); Franck TAILLEZ, Roquettes (FR); Jerome MENEGOTTO, Baziege (FR); Harikirshna TUMMA, Bangalore (IN); Lin WAN, Beijing (CN); Wei WANG, Beijing (CN); Rongfei YANG, Beijing (CN); Zheng Jane LI, Kendall Park, NJ (US); Ruiping WANG, Beijing (CN); Yue LU, Ningbo (CN)

(21) Appl. No.: 18/227,402

(22) Filed: Jul. 28, 2023

(51) Int. Cl. C07D 471/04

(52) U.S. Cl. CPC C07D 471/04; C07B 2200/13

(57) ABSTRACT

The present invention provides crystalline forms of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione (“Compound I”). Also provided are related pharmaceutical compositions, methods of preparation, and methods of treating hypertrophic cardiomyopathy (HCM), heart failure with preserved ejection fraction (HFpEF), diastolic dysfunction, left ventricular hypertrophy, and other cardiac diseases.

### CROSS REFERENCE TO RELATED APPLICATION

This application claims priority to PCT application no. PCT/CN2022/109030, filed Jul. 29, 2022, the entire contents of which are incorporated herein by reference.

### FIELD OF THE INVENTION

Provided herein are crystalline forms of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione (“Compound I”) as well as related pharmaceutical compositions, methods of preparation, and methods of treatment. Compound I has utility for the treatment of cardiac disorders, such as hypertrophic cardiomyopathy (HCM), heart failure, diastolic dysfunction, and left ventricular hypertrophy.

### BACKGROUND OF THE INVENTION

Hypertrophic cardiomyopathy (HCM) is a disease in which the heart muscle becomes thickened (hypertrophied), which can make it harder for the heart to pump blood. HCM is most commonly transmitted as an autosomal dominant trait, caused by mutations in genes encoding cardiac sarcomere proteins. U.S. Patent Appl. Publ. No. 2020/0165247 A1 discloses compounds that are designed to treat the underlying disruption in normal sarcomere function, with utility for the treatment of certain cardiac disorders, such as hypertrophic cardiomyopathy (HCM), heart failure, conditions associated with left ventricular hypertrophy, conditions associated with diastolic dysfunction, and/or symptoms thereof. One such compound is (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione. A crystalline form of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione is disclosed in US 2020/0165247 A1, and referred to therein as the Form 1 polymorph.

There is a need for pharmaceutically useful forms of such compounds including forms having properties appropriate for processing, manufacturing, storage stability, and/or usefulness as a drug. Accordingly, the discovery of a form that possesses some or all of these desired properties is vital to drug development.

### BRIEF SUMMARY OF THE INVENTION

The present invention provides crystalline forms of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione (“Compound I”).

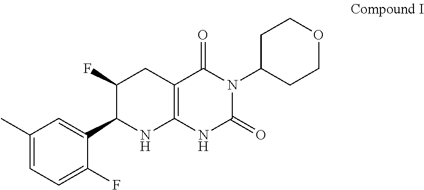 [SMILES: Cc1ccc(F)c([C@@H]2Nc3[nH]c(=O)n(C4CCOCC4)c(=O)c3C[C@@H]2F)c1]

Disclosed herein are polymorphs of Form A, B, C, D, E and F. In one aspect, disclosed herein is Compound I of Form B, C, D, E or F. In another aspect, disclosed herein is a mixture of two or more of Forms A, B, C, D, E, and F.

In another aspect, provided herein is a pharmaceutical composition comprising one or more crystalline forms as described herein and a pharmaceutically acceptable excipient.

In another aspect, provided herein is a method of making a crystalline form as described herein.

In another aspect, provided herein are methods of treating diastolic dysfunction, left ventricular hypertrophy, hypertrophic cardiomyopathy, heart failure with preserved ejection fraction, and other diseases. The methods include administering to a subject in need thereof an effective amount of a crystalline form or pharmaceutical composition as described herein.

### BRIEF DESCRIPTION OF THE DRAWINGS

FIG. 1 provides the X-ray powder diffraction (XRPD) pattern of Form A of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione (i.e., “Compound I”).

FIG. 2 provides the XRPD pattern for Compound I Form B

FIG. 3 provides the differential scanning calorimetry (DSC) thermogram for Compound I Form B.

FIG. 4 provides the thermogravimetric analysis (TGA) thermogram for Compound I Form B.

FIG. 5 provides the vapor sorption isotherm for Compound I Form B.

FIG. 6 provides the XRPD pattern for Compound I Form C.

FIG. 7 provides the DSC thermogram for Compound I Form C.

FIG. 8 provides the TGA thermogram for Compound I Form C.

FIG. 9 provides the XRPD pattern for Compound I Form D.

FIG. 10 provides the TGA thermogram for Compound I Form D.

FIG. 11 provides the XRPD pattern for Compound I Form E

FIG. 12 provides the DSC thermogram for Compound I Form E.

FIG. 13 provides the TGA thermogram for Compound I Form E.

FIG. 14 provides the XRPD pattern for Compound I Form F.

### DETAILED DESCRIPTION OF THE INVENTION

### I. Definitions

The terms “a,” “an,” or “the” as used herein not only include aspects with one member, but also include aspects with more than one member. For example, an embodiment including “an active agent and a pharmaceutically acceptable excipient” should be understood to present certain aspects with at least a second active agent, at least a second pharmaceutically acceptable excipient, or both. An embodiment including “an active agent” should be understood to present certain aspects with at least a second active agent, which may be of a different class.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” When the quantity “X” only includes whole-integer values (e.g., “X carbons”), “about X” indicates from (X−1) to (X+1). In this case, “about X” as used herein specifically indicates at least the values X, X−1, and X+1.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 110” is equivalent to “about 7%, about 9%, or about 11%.” “Agent” as used herein indicates a compound or mixture of compounds that, when added to a pharmaceutical composition, tend to produce a particular effect on the composition's properties. For example, a composition comprising a thickening agent is likely to be more viscous than an otherwise identical comparative composition that lacks the thickening agent.

As used herein, the phrase “effective amount” or “effective dose” means an amount sufficient to achieve the desired result and accordingly will depend on the ingredient and its desired result. Nonetheless, once the desired effect is known, determining the effective amount is within the skill of a person skilled in the art.

As used herein, the term “excipient” refers to a substance that aids the administration of an active agent to a subject. Pharmaceutical excipients useful in the present invention include, but are not limited to, binders, fillers, disintegrants, lubricants, coatings, sweeteners, flavors, and colors. One of skill in the art will recognize that other excipients can be useful in the present invention.

The term “pharmaceutical composition” as used herein refers to a product comprising a compound of the invention, an excipient as defined herein, and other optional ingredients in specified amounts, as well as any product which results directly or indirectly from combination of the specified ingredients in the specified amounts.

The term “pharmaceutically acceptable” means compatible with the treatment of animals, and in particular, humans.

The term “pharmaceutically acceptable salt” means a pharmaceutically acceptable acid addition salt or a pharmaceutically acceptable basic addition salt. The formation of a desired compound salt is achieved using standard techniques. For example, the neutral compound is treated with an acid or base in a suitable solvent and the formed salt is isolated by filtration, extraction, or any other suitable method. Pharmaceutically acceptable salts can be derived, for example, from mineral acids (hydrochloric acid, hydrobromic acid, phosphoric acid, and the like), organic acids (acetic acid, propionic acid, glutamic acid, citric acid and the like), and quaternary ammonium ions. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The term “subject” or “patient” as used herein includes all members of the animal kingdom, preferably mammals, and most preferably, humans.

The term “substantially” as applied to a composition or substance indicates at least 80% (w/w) identity as the designated substance, and preferably higher levels, such as at least 85%, 88%, 90%, 91%, 92%, 93%, 94%, 95%, 96%, 97%, 98%, or 99%.

As used herein, the terms “treat,” “treating” and “treatment” refer to any indicia of success in the treatment or amelioration of a pathology, injury, condition, or symptom related to a disease or disorder (for example a cardiac disorder having a pathophysiological feature of HCM), including any objective or subjective parameter such as abatement; remission; diminishing of symptoms; making the pathology, injury, condition, or symptom more tolerable to the patient; or decreasing the frequency or duration of the pathology, injury, condition, or symptom. Treatment or amelioration can be based on any objective or subjective parameter; including, e.g., the result of a physical examination.

The prefix “micro” as used herein can be alternatively abbreviated as “μ” or “u.” For example, micrograms are typically abbreviated as μg, but can alternatively be abbreviated as “ug.”

The term “polymorph” or “polymorphic form” refers to a crystalline form of a compound (or a salt, hydrate, or solvate thereof) in a particular crystal packing arrangement. All polymorphs have the same elemental composition. Different crystalline forms usually have different X-ray diffraction patterns, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point). The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a solid form substantially lacking three-dimensional order. In certain embodiments, an amorphous form of a solid is a solid form that is substantially not crystalline.

### II. Crystalline Forms

Disclosed herein are crystalline forms of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione (“Compound I”), depicted below. Compound I can be synthesized as disclosed in U.S. Patent Appl. Publ. No. 2020/0165247 A1.

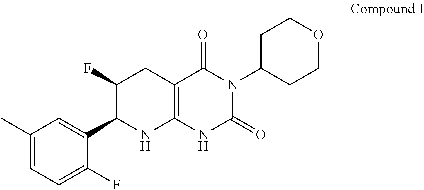 [SMILES: Cc1ccc(F)c([C@@H]2Nc3[nH]c(=O)n(C4CCOCC4)c(=O)c3C[C@@H]2F)c1]

Compound I polymorphs of Forms A, B, C, D, E and F are described herein. In another aspect, provided herein is a mixture of two or more of Forms A, B, C, D, E, and F.

### Form B

In one aspect, provided herein is the Form B polymorph of Compound I. Form B is an anhydrate form of the free base of Compound I. In another aspect, provided herein is a composition comprising Form B.

Form B is the most thermodynamically stable anhydrate form of Compound I under relevant conditions. Form B is stable at temperatures and conditions typical of pharmaceutical processes (e.g., 10-60° C.), resists transformation to other forms in certain solvents (including certain aqueous solvents and organic solvents), and may be crystallized from solvent at reasonable temperature (e.g., 10-60° C.). In competitive slurrying under certain conditions, Form B was the most thermodynamically stable anhydrate form. The stability of Form B is advantageous compared to other forms of Compound I that may transition between multiple polymorphic forms and mixtures thereof, a behavior that can result in inconsistency in the drug product. The ability to produce Form B by direct crystallization is advantageous in manufacturing to avoid additional processing steps and improve scalability, and also in allowing for control of physical properties. Form B has good manufacturability to make an ordered crystal structure of suitable purity. Form B is non-hygroscopic. The non-hygroscopic nature of Form B is advantageous in that it avoids water uptake, which can lead to issues with stability and issues with providing an accurate dosage due to uncertainty as to percent of water in a pharmaceutical composition. The non-hygroscopicity of Form B is also beneficial for manufacturability and processability of both drug substance and drug product. Form B has a high melting point and relatively good solid state thermostability, as evidenced by the DSC thermogram for Form B. Form B has minimum weight loss before melt and no concern of desolvation or dehydration, as evidenced by the TGA thermogram for Form B. These properties also contribute to the stability of Form B and the resulting consistency of a drug product comprising Form B.

In some embodiments, Form B is characterized by an X-ray powder diffraction pattern, obtained by irradiation with Cu-Kα at room temperature, comprising at least three peaks, or at least four peaks, selected from the group consisting of 5.5, 7.1, 9.3, 16.5, and 19.0 °2θ±0.2 °2θ.

In some embodiments, Form B is characterized by an X-ray powder diffraction pattern, obtained by irradiation with Cu-Kα at room temperature, comprising peaks at 5.5, 9.3, and 19.0 °2θ±0.2 °2θ.

In some embodiments, Form B is characterized by an X-ray powder diffraction pattern, obtained by irradiation with Cu-Kα at room temperature, comprising peaks at 9.3, 16.5, and 19.0 °2θ±0.2 °2θ.

In some embodiments, Form B is characterized by an X-ray powder diffraction pattern, obtained by irradiation with Cu-Kα at room temperature, comprising peaks at 5.5, 9.3, 16.5, and 19.0 °2θ±0.2 °2θ.

In some embodiments, Form B is characterized by an X-ray powder diffraction pattern, obtained by irradiation with Cu-Kα at room temperature, comprising peaks at 5.5, 7.1, 9.3, and 19.0 °2θ±0.2 °2θ.

In some embodiments, Form B is characterized by an X-ray powder diffraction pattern, obtained by irradiation with Cu-Kα at room temperature, comprising peaks at 5.5, 7.1, 9.3, 16.5, 19.0, and 22.3 °2θ±0.2 °2θ.

In some embodiments, Form B is characterized by an X-ray powder diffraction pattern, obtained by irradiation with Cu-Kα at room temperature, comprising peaks at 5.5, 7.1, 9.3, 16.5, 19.0, and 22.3 °2θ±0.1 °2θ.

In some embodiments, Form B is characterized by an X-ray powder diffraction pattern, obtained by irradiation with Cu-Kα at room temperature, comprising a peak at 5.5 °2θ±0.2 °2θ and at least four peaks, at least five peaks, at least six peaks, at least seven peaks, or at least eight peaks selected from the group consisting of 7.1, 8.7, 9.3, 13.8, 16.2, 16.5, 17.2, 19.0, and 22.3 °2θ±0.2 °2θ.

In some embodiments, Form B is characterized by an X-ray powder diffraction pattern, obtained by irradiation with Cu-Kα at room temperature, comprising peaks at 5.5, 7.1, 8.7, 9.3, 13.8, 16.2, 16.5, 17.2, 19.0, and 22.3 °2θ±0.2 °2θ.

In some embodiments, Form B is characterized by an X-ray powder diffraction pattern, obtained by irradiation with Cu-Kα at room temperature, comprising peaks at 5.5, 7.1, 8.7, 9.3, 13.8, 16.2, 16.5, 17.2, 19.0, and 22.3 °2θ±0.1 °2θ.

In some embodiments, Form B is characterized by an X-ray powder diffraction pattern, obtained by irradiation with Cu-Kα at room temperature, comprising peaks at 5.5, 7.1, 8.7, 9.3, 13.8, 15.8, 16.2, 16.5, 17.2, 18.5, 19.0, 19.3, 22.3, 23.1, 23.4, 23.7, 26.2, 27.3, 27.9, and 30.1 °2θ±0.2 °2θ.

In some embodiments, Form B is characterized by an X-ray powder diffraction pattern, obtained by irradiation with Cu-Kα at room temperature, comprising a peak at 5.5 °2θ±0.2 °2θ and at least two peaks, or at least three peaks, selected from the group consisting of 7.1, 9.3, 16.5, and 19.0 °2θ±0.2 °2θ.

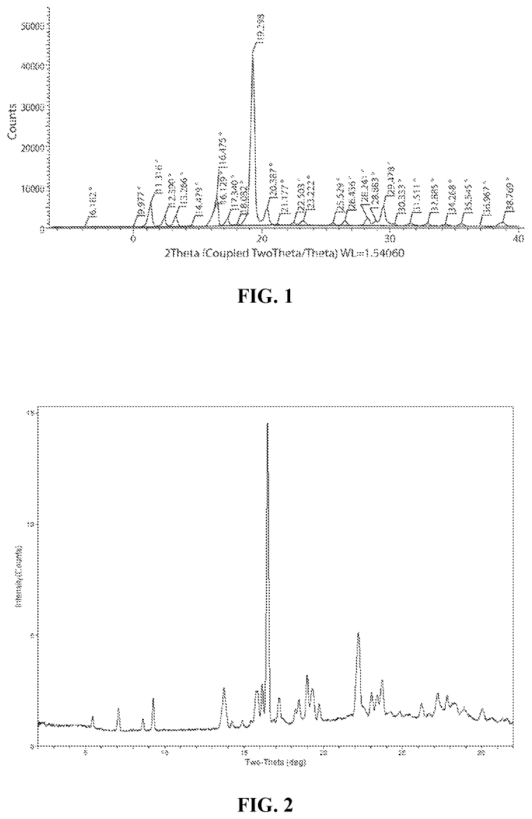

In some embodiments, Form B is characterized by an X-ray powder diffraction pattern, obtained by irradiation with Cu-Kα at room temperature, substantially as shown in FIG. 2.

Table 1 lists the observed X-ray powder diffraction peaks from FIG. 2.

```
TABLE 1
Observed X-ray Powder Diffraction Peaks for Form B (°2θ)
  5.5 ± 0.2
  7.1 ± 0.2
  8.7 ± 0.2
  9.3 ± 0.2
13.8 ± 0.2
15.8 ± 0.2
16.2 ± 0.2
16.5 ± 0.2
17.2 ± 0.2
18.5 ± 0.2
19.0 ± 0.2
19.3 ± 0.2
22.3 ± 0.2
23.1 ± 0.2
23.4 ± 0.2
23.7 ± 0.2
26.2 ± 0.2
27.3 ± 0.2
27.9 ± 0.2
30.1 ± 0.2
```

In some embodiments, Form B is characterized by a DSC thermogram comprising an endotherm onset at about 307° C.

In some embodiments, Form B is characterized by (i) an X-ray powder diffraction pattern, obtained by irradiation with Cu-Kα at room temperature, comprising peaks at 5.5, 9.3, and 19.0 °2θ±0.2 °2θ and (ii) a DSC thermogram comprising an endotherm onset at about 307° C.

In some embodiments, Form B is characterized by a DSC thermogram substantially as shown in FIG. 3.

In some embodiments, Form B is characterized by exhibiting a negligible weight loss until decomposition as measured by TGA.

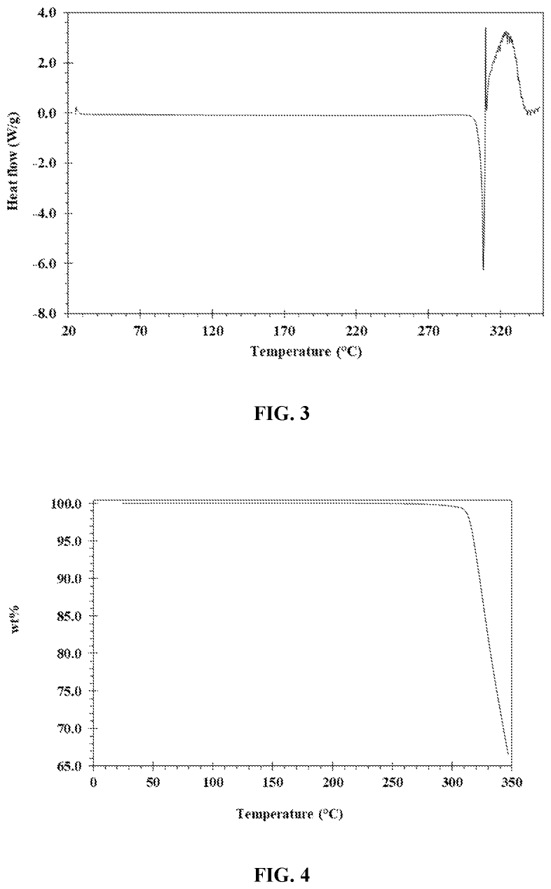

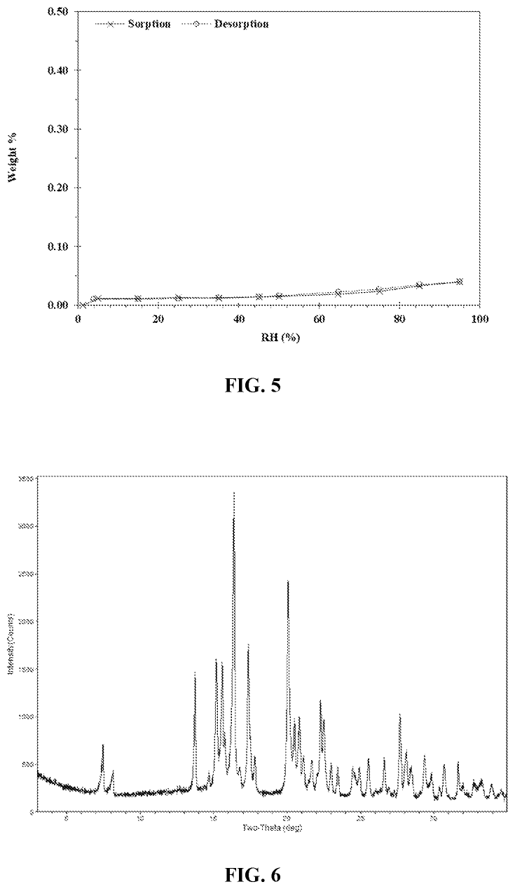

In some embodiments, Form B is characterized by a TGA thermogram substantially as shown in FIG. 4.

In some embodiments, Form B is characterized by a weight percent gain of less than 0.05% up to 95% RH as measured by vapor sorption analyzer (e.g., TA Instrument VTI-SA+). Vapor sorption analysis showed that Form B is non-hygroscopic.

In some embodiments, Form B is characterized by a triclinic crystal system with a P1 space group.

In some embodiments, Form B is characterized by (i) an X-ray powder diffraction pattern, obtained by irradiation with Cu-Kα at room temperature, comprising peaks at 5.5, 9.3, and 19.0 °2θ±0.2 °2θ and (ii) a triclinic crystal system with a P1 space group.

In some embodiments, Form B is characterized by (i) an X-ray powder diffraction pattern, obtained by irradiation with Cu-Kα at room temperature, comprising peaks at 5.5, 9.3, and 19.0 °2θ±0.2 °2θ; (ii) a triclinic crystal system with a P1 space group; and (iii) a DSC thermogram comprising an endotherm onset at about 307° C.

In some embodiments, Form B is characterized by unit cell dimensions, at a temperature of 100 Kelvin, of a=6.74±0.10 Å, b=12.74±0.10 Å, c=15.99±0.10 Å, α=83.9±1.0°, β=80.0±1.0°, and γ=75.1±1.0°.

Table 2 provides the single crystal x-ray analysis data for Form B.

TABLE 2
Single Crystal X-ray Analysis for Form B

| | | |
|---|---|---|
| T/K | 100 | |
| Crystal System | triclinic | |
| Space Group | P1 | |
| Unit Cell | | |
| Dimensions: | | |
| | a = 6.74 ± 0.10 Å | α = 83.9 ± 1.0° |
| | b = 12.74 ± 0.10 Å | β = 80.0 ± 1.0° |
| | c = 15.99 ± 0.10 Å | γ = 75.1 ± 1.0° |
| Volume | 1303(20) Å³ | |
| Z | 3 | |
| D_{calc.}/g cm⁻³ | 1.443 | |

In some embodiments, Form B is substantially pure, i.e., substantially free of other solid forms of Compound I (including amorphous Compound I and other polymorphs of Compound I).

In some embodiments, provided herein is a composition comprising Compound I Form B, wherein the composition is at least 90% Form B by weight. In some embodiments, composition is at least 91%, at least 92%, at least 93%, at least 94%, at least 95%, at least 96%, at least 97%, at least 98%, at least 99% or at least 99.5% Form B by weight. In some embodiments, the ratio (by weight) of Form B to other solid forms of Compound I in the composition is at least 90:10, at least 95:5, at least 98:2, at least 99:1, or at least 99.5:0.5.

### Form C

In one aspect, provided herein is the Form C polymorph of Compound I. Form C is an anhydrate form of the free base of Compound I. In another aspect, provided herein is a composition comprising Form C.

Form C has a high melting point and relatively good solid state thermostability, as evidenced by the DSC thermogram for Form C. Form C has minimum weight loss before melt and no concern of desolvation or dehydration, as evidenced by the TGA thermogram for Form C. These properties may contribute to the consistency of a drug product comprising Form C. Form C may be crystallized from solvent at reasonable temperature. The ability to produce Form C by direct crystallization is advantageous in manufacturing to avoid additional processing steps and improve scalability, and also in allowing for control of physical properties.

In some embodiments, Form C is characterized by an X-ray powder diffraction pattern, obtained by irradiation with Cu-Kα at room temperature, comprising at least three peaks, at least four peaks, or at least five peaks selected from the group consisting of 7.5, 13.8, 16.4, 17.4, 20.1, and 27.7 °2θ±0.2 °2θ.

In some embodiments, Form C is characterized by an X-ray powder diffraction pattern, obtained by irradiation with Cu-Kα at room temperature, comprising peaks at 7.5, 13.8, and 16.4 °2θ±0.2 °2θ.

In some embodiments, Form C is characterized by an X-ray powder diffraction pattern, obtained by irradiation with Cu-Kα at room temperature, comprising peaks at 7.5, 16.4, and 20.1 °2θ±0.2 °2θ

In some embodiments, Form C is characterized by an X-ray powder diffraction pattern, obtained by irradiation with Cu-Kα at room temperature, comprising peaks at 7.5, 16.4, and 27.7 °2θ±0.2 °2θ.

In some embodiments, Form C is characterized by an X-ray powder diffraction pattern, obtained by irradiation with Cu-Kα at room temperature, comprising peaks at 7.5, 13.8, 16.4, 17.4, 20.1, and 27.7 °2θ±0.2 °2θ.

In some embodiments, Form C is characterized by an X-ray powder diffraction pattern, obtained by irradiation with Cu-Kα at room temperature, comprising peaks at 7.5, 13.8, 16.4, 17.4, 20.1, and 27.7 °2θ±0.1 °2θ.

In some embodiments, Form C is characterized by an X-ray powder diffraction pattern, obtained by irradiation with Cu-Kα at room temperature, comprising at least four peaks, at least five peaks, at least six peaks, at least seven peaks, at least eight peaks, or at least nine peaks selected from the group consisting of 7.5, 8.2, 13.8, 15.2, 15.6, 16.4, 17.4, 20.1, 22.3, and 27.7 °2θ±0.2 °2θ.

In some embodiments, Form C is characterized by an X-ray powder diffraction pattern, obtained by irradiation with Cu-Kα at room temperature, comprising peaks at 7.5, 8.2, 13.8, 15.2, 15.6, 16.4, 17.4, 20.1, 22.3, and 27.7 °2θ±0.2 °2θ.

In some embodiments, Form C is characterized by an X-ray powder diffraction pattern, obtained by irradiation with Cu-Kα at room temperature, comprising peaks at 7.5, 8.2, 13.8, 15.2, 15.6, 16.4, 17.4, 20.1, 22.3, and 27.7 °2θ±0.1 °2θ.

In some embodiments, Form C is characterized by an X-ray powder diffraction pattern, obtained by irradiation with Cu-Kα at room temperature, comprising peaks at 7.5, 8.2, 13.8, 14.7, 15.2, 15.6, 16.4, 17.4, 17.8, 20.1, 20.5, 20.9, 21.2, 21.7, 22.3, 22.6, 23.0, 23.5, 24.5, 25.0, 25.6, 26.7, 27.7, 28.2, 28.5, 29.4, 29.9, 30.8, and 31.7 °2θ±0.2 °2θ.

In some embodiments, Form C is characterized by an X-ray powder diffraction pattern, obtained by irradiation with Cu-Kα at room temperature, substantially as shown in FIG. 6.

Table 3 lists the observed X-ray powder diffraction peaks from FIG. 6.

```
TABLE 3
Observed X-ray Powder Diffraction Peaks for Form C (°2θ)
  7.5 ± 0.2
  8.2 ± 0.2
13.8 ± 0.2
14.7 ± 0.2
15.2 ± 0.2
15.6 ± 0.2
16.4 ± 0.2
17.4 ± 0.2
17.8 ± 0.2
20.1 ± 0.2
20.5 ± 0.2
20.9 ± 0.2
21.2 ± 0.2
21.7 ± 0.2
22.3 ± 0.2
22.6 ± 0.2
23.0 ± 0.2
23.5 ± 0.2
24.5 ± 0.2
25.0 ± 0.2
25.6 ± 0.2
26.7 ± 0.2
27.7 ± 0.2
28.2 ± 0.2
28.5 ± 0.2
29.4 ± 0.2
29.9 ± 0.2
30.8 ± 0.2
31.7 ± 0.2
```

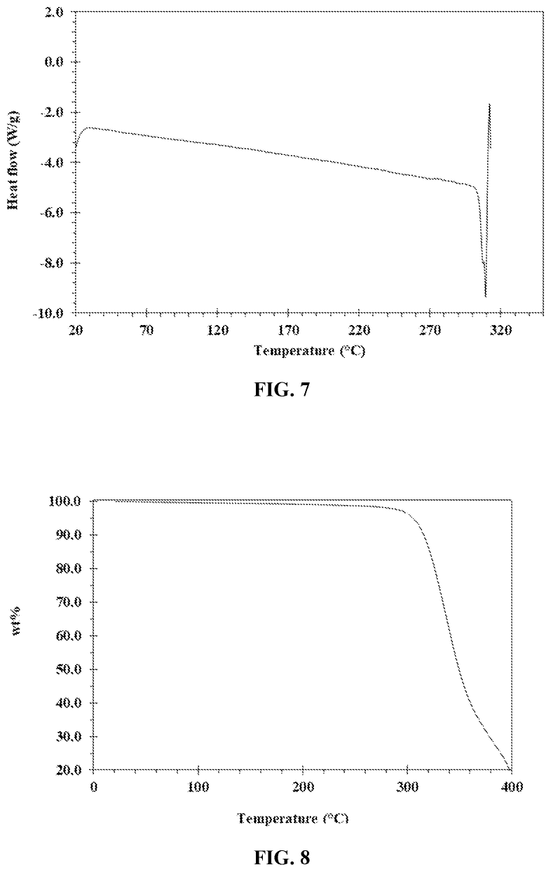

In some embodiments, Form C is characterized by a DSC thermogram comprising an endotherm onset at about 306° C. In some embodiments, Form C is characterized by a DSC thermogram substantially as shown in FIG. 7.

In some embodiments, Form C is characterized by exhibiting a negligible weight loss until decomposition as measured by TGA. In some embodiments, Form C is characterized by a TGA thermogram substantially as shown in FIG. 8.

In some embodiments, Form C is substantially pure, i.e., substantially free of other solid forms of Compound I.

In some embodiments, provided herein is a composition comprising Compound I Form C, wherein the composition is at least 90% Form C by weight. In some embodiments, composition is at least 91%, at least 92%, at least 93%, at least 94%, at least 95%, at least 96%, at least 97%, at least 98%, at least 99% or at least 99.5% Form C by weight. In some embodiments, the ratio (by weight) of Form C to other solid forms of Compound I in the composition is at least 90:10, at least 95:5, at least 98:2, at least 99:1, or at least 99.5:0.5.

### Form D

In one aspect, provided herein is the Form D polymorph of Compound I. Form D is an anhydrate form of the free base of Compound I. In another aspect, provided herein is a composition comprising Form D.

Form D has minimum weight loss before melt and no concern of desolvation or dehydration, as evidenced by the TGA thermogram for Form D. These properties may contribute to the consistency of a drug product comprising Form D.

In some embodiments, Form D is characterized by an X-ray powder diffraction pattern, obtained by irradiation with Cu-Kα at room temperature, comprising at least three peaks, at least four peaks, at least five peaks, or at least six peaks selected from the group consisting of 11.1, 15.0, 18.3, 19.8, 20.2, 22.5, and 25.9 °2θ±0.2 °2θ.

In some embodiments, Form D is characterized by an X-ray powder diffraction pattern, obtained by irradiation with Cu-Kα at room temperature, comprising peaks at 11.1, 15.0, and 25.9 °2θ±0.2 °2θ.

In some embodiments, Form D is characterized by an X-ray powder diffraction pattern, obtained by irradiation with Cu-Kα at room temperature, comprising peaks at 11.1, 15.0, 19.8 and 25.9 °2θ±0.2 °2θ.

In some embodiments, Form D is characterized by an X-ray powder diffraction pattern, obtained by irradiation with Cu-Kα at room temperature, comprising peaks at 11.1, 15.0, 18.3, 19.8, 20.2, 22.5, and 25.9 °2θ±0.2 °2θ.

In some embodiments, Form D is characterized by an X-ray powder diffraction pattern, obtained by irradiation with Cu-Kα at room temperature, comprising peaks at 11.1, 15.0, 18.3, 19.8, 20.2, 22.5, and 25.9 °2θ±0.1 °2θ.

In some embodiments, Form D is characterized by an X-ray powder diffraction pattern, obtained by irradiation with Cu-Kα at room temperature, comprising at least four peaks, at least five peaks, at least six peaks, at least seven peaks, at least eight peaks, or at least nine peaks selected from the group consisting of 11.1, 15.0, 18.3, 19.8, 20.2, 22.5, 23.0, 25.0, 25.9, and 28.2 °2θ±0.2 °2θ.

In some embodiments, Form D is characterized by an X-ray powder diffraction pattern, obtained by irradiation with Cu-Kα at room temperature, comprising peaks at 11.1, 15.0, 18.3, 19.8, 20.2, 22.5, 23.0, 25.0, 25.9, and 28.2 °2θ±0.2 °2θ.

In some embodiments, Form D is characterized by an X-ray powder diffraction pattern, obtained by irradiation with Cu-Kα at room temperature, comprising peaks at 11.1, 15.0, 18.3, 19.8, 20.2, 22.5, 23.0, 25.0, 25.9, and 28.2 °2θ±0.1 °2θ.

In some embodiments, Form D is characterized by an X-ray powder diffraction pattern, obtained by irradiation with Cu-Kα at room temperature, comprising peaks at 11.1, 14.0, 15.0, 15.4, 18.3, 19.3, 19.8, 20.2, 20.7, 22.5, 23.0, 25.0, 25.9, 26.7, 27.4, 28.2, 30.1, and 31.8 °2θ±0.2 °2θ.

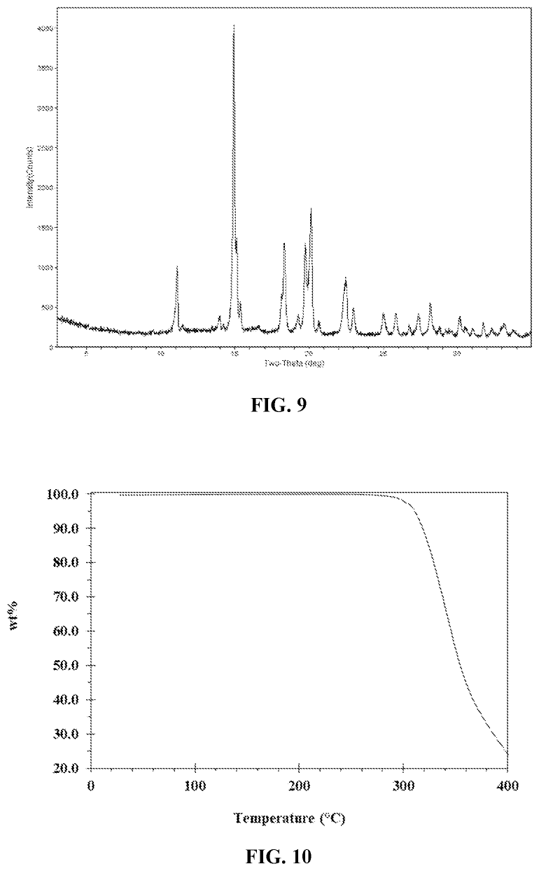

In some embodiments, Form D is characterized by an X-ray powder diffraction pattern, obtained by irradiation with Cu-Kα at room temperature, substantially as shown in FIG. 9.

Table 4 lists the observed X-ray powder diffraction peaks from FIG. 9.

TABLE 4
Observed X-ray Powder Diffraction Peaks for Form D (°2θ)
11.1 ± 0.2
14.0 ± 0.2
15.0 ± 0.2
15.4 ± 0.2
18.3 ± 0.2
19.3 ± 0.2
19.8 ± 0.2
20.2 ± 0.2
20.7 ± 0.2
22.5 ± 0.2
23.0 ± 0.2
25.0 ± 0.2
25.9 ± 0.2
26.7 ± 0.2
27.4 ± 0.2
28.2 ± 0.2
30.1 ± 0.2
31.8 ± 0.2

In some embodiments, Form D is characterized by exhibiting a negligible weight loss until decomposition as measured by TGA. In some embodiments, Form D is characterized by a TGA thermogram substantially as shown in FIG. 10.

In some embodiments, Form D is substantially pure, i.e., substantially free of other solid forms of Compound I.

In some embodiments, provided herein is a composition comprising Compound I Form D, wherein the composition is at least 90% Form D by weight. In some embodiments, composition is at least 91%, at least 92%, at least 93%, at least 94%, at least 95%, at least 96%, at least 97%, at least 98%, at least 99% or at least 99.5% Form D by weight. In some embodiments, the ratio (by weight) of Form D to other solid forms of Compound I in the composition is at least 90:10, at least 95:5, at least 98:2, at least 99:1, or at least 99.5:0.5.

### Form E

In one aspect, provided herein is the Form E polymorph of Compound I. Form E is an anhydrate form of the free base of Compound I. In another aspect, provided herein is a composition comprising Form E.

Form E may be crystallized from solvent at reasonable temperature (e.g., 10-60° C.). The ability to produce Form E by direct crystallization is advantageous in manufacturing to avoid additional processing steps and improve scalability, and also in allowing for control of physical properties. Form E has minimum weight loss before melt and no concern of desolvation or dehydration, as evidenced by the TGA thermogram for Form E. Form E has a high melting point and relatively good solid state thermostability, as evidenced by the DSC thermogram for Form E. These properties may contribute to the consistency of a drug product comprising Form E.

In some embodiments, Form E is characterized by an X-ray powder diffraction pattern, obtained by irradiation with Cu-Kα at room temperature, comprising at least three peaks, or at least four peaks, selected from the group consisting of 4.1, 8.6, 16.5, 17.7, and 23.2 °2θ±0.2 °2θ.

In some embodiments, Form E is characterized by an X-ray powder diffraction pattern, obtained by irradiation with Cu-Kα at room temperature, comprising peaks at 8.6, 17.7, and 23.2 °2θ±0.2 °2θ.

In some embodiments, Form E is characterized by an X-ray powder diffraction pattern, obtained by irradiation with Cu-Kα at room temperature, comprising peaks at 4.1, 8.6, and 16.5 °2θ±0.2 °2θ.

In some embodiments, Form E is characterized by an X-ray powder diffraction pattern, obtained by irradiation with Cu-Kα at room temperature, comprising peaks at 4.1, 8.6, and 17.7 °2θ±0.2 °2θ.

In some embodiments, Form E is characterized by an X-ray powder diffraction pattern, obtained by irradiation with Cu-Kα at room temperature, comprising peaks at 8.6, 16.5, 17.7, and 23.2 °2θ±0.2 °2θ.

In some embodiments, Form E is characterized by an X-ray powder diffraction pattern, obtained by irradiation with Cu-Kα at room temperature, comprising peaks at 8.6, 17.7, 22.3, and 23.2 °2θ±0.2 °2θ.

In some embodiments, Form E is characterized by an X-ray powder diffraction pattern, obtained by irradiation with Cu-Kα at room temperature, comprising peaks at 4.1, 8.6, 16.5, 17.7, 22.3, and 23.2 °2θ±0.2 °2θ.

In some embodiments, Form E is characterized by an X-ray powder diffraction pattern, obtained by irradiation with Cu-Kα at room temperature, comprising peaks at 4.1, 8.6, 16.5, 17.7, 22.3, and 23.2 °2θ±0.1 °2θ.

In some embodiments, Form E is characterized by an X-ray powder diffraction pattern, obtained by irradiation with Cu-Kα at room temperature, comprising at least four peaks, at least five peaks, at least six peaks, at least seven peaks, at least eight peaks, or at least nine peaks selected from the group consisting of 4.1, 7.2, 8.6, 13.6, 15.7, 16.5, 17.7, 19.3, 22.3, and 23.2 °2θ±0.2 °2θ.

In some embodiments, Form E is characterized by an X-ray powder diffraction pattern, obtained by irradiation with Cu-Kα at room temperature, comprising peaks at 4.1, 7.2, 8.6, 13.6, 15.7, 16.5, 17.7, 19.3, 22.3, and 23.2 °2θ±0.2 °2θ.

In some embodiments, Form E is characterized by an X-ray powder diffraction pattern, obtained by irradiation with Cu-Kα at room temperature, comprising peaks at 4.1, 7.2, 8.6, 13.6, 15.7, 16.5, 17.7, 19.3, 22.3, and 23.2 °2θ±0.1 °2θ.

In some embodiments, Form E is characterized by an X-ray powder diffraction pattern, obtained by irradiation with Cu-Kα at room temperature, comprising peaks at 4.1, 7.2, 7.9, 8.6, 13.6, 14.3, 15.3, 15.7, 16.1, 16.5, 17.3, 17.7, 18.3, 18.7, 19.3, 21.1, 21.7, 22.3, 23.2, 26.4, 27.6, 28.5, 28.8, and 30.2 °2θ±0.2 °2θ.

In some embodiments, Form E is characterized by an X-ray powder diffraction pattern, obtained by irradiation with Cu-Kα at room temperature, comprising a peak at 4.1 °2θ±0.2 °2θ and at least two peaks, or at least three peaks, selected from the group consisting of 8.6, 16.5, 17.7, and 23.2 °2θ±0.2 °2θ.

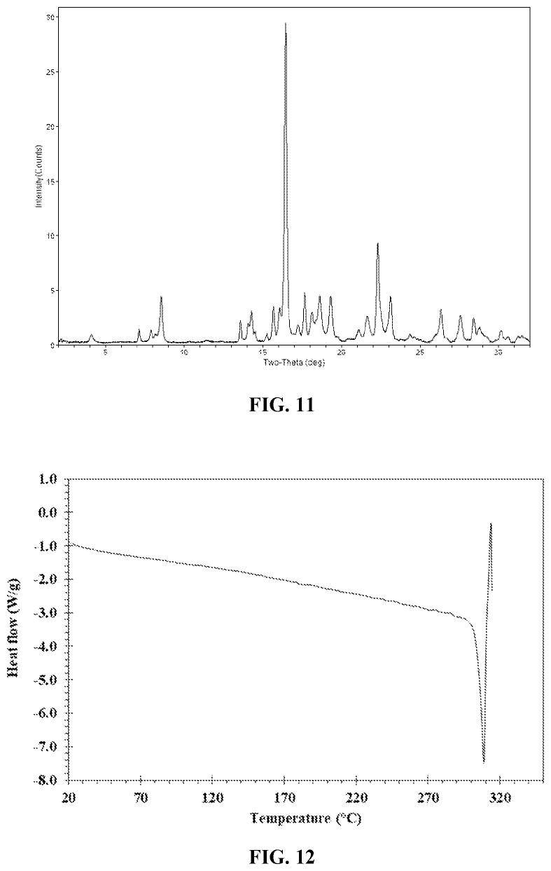

In some embodiments, Form E is characterized by an X-ray powder diffraction pattern, obtained by irradiation with Cu-Kα at room temperature, substantially as shown in FIG. 11.

Table 5 lists the observed X-ray powder diffraction peaks from FIG. 11.

```
TABLE 5
Observed X-ray Powder Diffraction Peaks for Form E (°2θ)
  4.1 ± 0.2
  7.2 ± 0.2
  7.9 ± 0.2
  8.6 ± 0.2
13.6 ± 0.2
14.3 ± 0.2
15.3 ± 0.2
15.7 ± 0.2
16.1 ± 0.2
16.5 ± 0.2
17.3 ± 0.2
17.7 ± 0.2
18.3 ± 0.2
18.7 ± 0.2
19.3 ± 0.2
21.1 ± 0.2
21.7 ± 0.2
22.3 ± 0.2
23.2 ± 0.2
26.4 ± 0.2
27.6 ± 0.2
28.5 ± 0.2
28.8 ± 0.2
30.2 ± 0.2
```

In some embodiments, Form E is characterized by a DSC thermogram comprising an endotherm onset at about 304° C. In some embodiments, Form E is characterized by a DSC thermogram substantially as shown in FIG. 12.

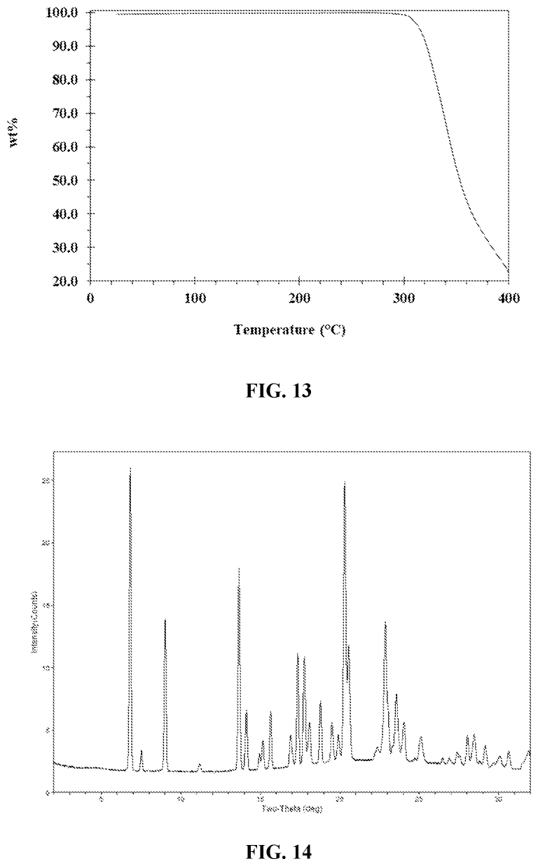

In some embodiments, Form E is characterized by exhibiting a negligible weight loss until decomposition as measured by TGA. In some embodiments, Form E is characterized by a TGA thermogram substantially as shown in FIG. 13.

In some embodiments, Form E is characterized by (i) an X-ray powder diffraction pattern, obtained by irradiation with Cu-Kα at room temperature, comprising at peaks at 8.6, 17.7, and 23.2 °2θ±0.2 °2θ and (ii) a DSC thermogram comprising an endotherm onset at about 304° C.

In some embodiments, Form E is characterized by (i) an X-ray powder diffraction pattern, obtained by irradiation with Cu-Kα at room temperature, comprising at peaks at 8.6, 17.7, and 23.2 °2θ±0.2 °2θ and (ii) a monoclinic crystal system with a C2 space group.

In some embodiments, Form E is characterized by (i) an X-ray powder diffraction pattern, obtained by irradiation with Cu-Kα at room temperature, comprising at peaks at 8.6, 17.7, and 23.2 °2θ±0.2 °2θ, (ii) a monoclinic crystal system with a C2 space group, and (iii) a DSC thermogram comprising an endotherm onset at about 304° C.

In some embodiments, Form E is characterized by a monoclinic crystal system with a C2 space group. In some embodiments, Form E is characterized by unit cell dimensions, at a temperature of 302 Kelvin, of a=24.75±0.10 Å, b=6.73±0.10 Å, c=21.64±0.10 Å, α=90.0±1.0°, β=95.7±1.0°, and γ=90.0±1.0°.

Table 6 shows the single crystal x-ray analysis data for Form E.

TABLE 6
Single Crystal X-ray Analysis for Form E

| | | |
|---|---|---|
| T/K | 302 | |
| Crystal System | Monoclinic | |
| Space Group | C2 | |
| Unit Cell | | |
| Dimensions: | | |
| | a = 24.75 ± 0.10 Å | α = 90.0 ± 1.0° |
| | b = 6.73 ± 0.10 Å | β = 95.7 ± 1.0° |
| | c = 21.64 ± 0.10 Å | γ = 90.0 ± 1.0° |
| Volume | 3585(20) Å³ | |
| Z | 8 | |
| D_{calc.}/g cm⁻³ | 1.398 | |

In some embodiments, Form E is substantially pure, i.e., substantially free of other solid forms of Compound I.

In some embodiments, provided herein is a composition comprising Compound I Form E, wherein the composition is at least 90% Form E by weight. In some embodiments, composition is at least 91%, at least 92%, at least 93%, at least 94%, at least 95%, at least 96%, at least 97%, at least 98%, at least 99% or at least 99.5% Form E by weight. In some embodiments, the ratio (by weight) of Form E to other solid forms of Compound I in the composition is at least 90:10, at least 95:5, at least 98:2, at least 99:1, or at least 99.5:0.5.

### Form F

In one aspect, provided herein is the Form F polymorph of Compound I. Form F is a solvated form of the free base of Compound I. Form F can be produced from variety of organic solvents, including certain ethers (e.g. THF), acetates (e.g. isopropyl acetate), alcohols (e.g. methanol), aromatic solvents (e.g. toluene), ketones (e.g. acetone). In another aspect, provided herein is a composition comprising Form F.

Form F is a crystalline form and could be used for purification. Form F can be used to make a variety of forms, including anhydrous forms, and therefore is useful for manufacturing purposes.

In some embodiments, Form F is characterized by an X-ray powder diffraction pattern, obtained by irradiation with Cu-Kα at room temperature, comprising at least three peaks, or at least four peaks, selected from the group consisting of 6.8, 9.0, 13.7, 14.2, and 20.3 °2θ±0.2 °2θ.

In some embodiments, Form F is characterized by an X-ray powder diffraction pattern, obtained by irradiation with Cu-Kα at room temperature, comprising peaks at 6.8, 9.0, and 14.2 °2θ±0.2 °2θ.

In some embodiments, Form F is characterized by an X-ray powder diffraction pattern, obtained by irradiation with Cu-Kα at room temperature, comprising peaks at 6.8, 9.0, and 20.3 °2θ±0.2 °2θ.

In some embodiments, Form F is characterized by an X-ray powder diffraction pattern, obtained by irradiation with Cu-Kα at room temperature, comprising peaks at 6.8, 9.0, 13.7, and 14.2 °2θ±0.2 °2θ.

In some embodiments, Form F is characterized by an X-ray powder diffraction pattern, obtained by irradiation with Cu-Kα at room temperature, comprising peaks at 6.8, 9.0, 14.2, and 20.3 °2θ±0.2 °2θ.

In some embodiments, Form F is characterized by an X-ray powder diffraction pattern, obtained by irradiation with Cu-Kα at room temperature, comprising peaks at 9.0, 13.7, 14.2, and 20.3 °2θ±0.2 °2θ.

In some embodiments, Form F is characterized by an X-ray powder diffraction pattern, obtained by irradiation with Cu-Kα at room temperature, comprising peaks at 6.8, 9.0, 13.7, 14.2, 20.3, and 22.9 °2θ±0.2 °2θ.

In some embodiments, Form F is characterized by an X-ray powder diffraction pattern, obtained by irradiation with Cu-Kα at room temperature, comprising peaks at 6.8, 9.0, 13.7, 14.2, 20.3, and 22.9 °2θ±0.1 °2θ.

In some embodiments, Form F is characterized by an X-ray powder diffraction pattern, obtained by irradiation with Cu-Kα at room temperature, comprising at least four peaks, at least five peaks, at least six peaks, at least seven peaks, at least eight peaks, or at least nine peaks selected from the group consisting of 6.8, 9.0, 13.7, 14.2, 15.7, 17.4, 17.8, 18.8, 20.3, and 22.9 °2θ±0.2 °2θ.

In some embodiments, Form F is characterized by an X-ray powder diffraction pattern, obtained by irradiation with Cu-Kα at room temperature, comprising peaks at 6.8, 9.0, 13.7, 14.2, 15.7, 17.4, 17.8, 18.8, 20.3, and 22.9 °2θ±0.2 °2θ.

In some embodiments, Form F is characterized by an X-ray powder diffraction pattern, obtained by irradiation with Cu-Kα at room temperature, comprising peaks at 6.8, 9.0, 13.7, 14.2, 15.7, 17.4, 17.8, 18.8, 20.3, and 22.9 °2θ±0.1 °2θ.

In some embodiments, Form F is characterized by an X-ray powder diffraction pattern, obtained by irradiation with Cu-Kα at room temperature, comprising peaks at 6.8, 7.5, 9.0, 11.2, 13.7, 14.2, 15.2, 15.7, 16.9, 17.4, 17.8, 18.1, 18.8, 19.5, 19.9, 20.3, 20.6, 22.4, 22.9, 23.6, 24.1, 25.1, 27.4, 28.1, 28.5, 29.2, 30.1, and 30.7 °2θ±0.2 °2θ.

In some embodiments, Form F is characterized by an X-ray powder diffraction pattern, obtained by irradiation with Cu-Kα at room temperature, substantially as shown in FIG. 14.

Table 7 lists the observed X-ray powder diffraction peaks from FIG. 14.

```
TABLE 7
Observed X-ray Powder Diffraction Peaks for Form F (°2θ)
  6.8 ± 0.2
  7.5 ± 0.2
  9.0 ± 0.2
11.2 ± 0.2
13.7 ± 0.2
14.2 ± 0.2
15.2 ± 0.2
15.7 ± 0.2
16.9 ± 0.2
17.4 ± 0.2
17.8 ± 0.2
18.1 ± 0.2
18.8 ± 0.2
19.5 ± 0.2
19.9 ± 0.2
20.3 ± 0.2
20.6 ± 0.2
22.4 ± 0.2
22.9 ± 0.2
23.6 ± 0.2
24.1 ± 0.2
25.1 ± 0.2
27.4 ± 0.2
28.1 ± 0.2
28.5 ± 0.2
29.2 ± 0.2
30.1 ± 0.2
30.7 ± 0.2
```

In some embodiments, Form F is substantially pure, i.e., substantially free of other solid forms of Compound I.

In some embodiments, provided herein is a composition comprising Compound I Form F, wherein the composition is at least 90% Form F by weight. In some embodiments, composition is at least 91%, at least 92%, at least 93%, at least 94%, at least 95%, at least 96%, at least 97%, at least 98%, at least 99% or at least 99.5% Form F by weight. In some embodiments, the ratio (by weight) of Form F to other solid forms of Compound I in the composition is at least 90:10, at least 95:5, at least 98:2, at least 99:1, or at least 99.5:0.5.

### III. Methods of Preparation

In one aspect, provided herein is a method of making the Form B polymorph of Compound I, the method comprising dissolving Compound I in tetrahydrofuran (THF), removing the THF by rotary evaporation, slurrying the resulting solids in ethanol, thermally cycling the resulting slurry, filtering the slurry, and drying to isolate the solids of Form B.

In another aspect, provided herein is a method of making the Form B polymorph of Compound I, the method comprising slurrying Form A of Compound I in ethanol, thermally cycling the resulting slurry, filtering the slurry and drying, followed by slurrying the resulting solids in ethanol at elevated temperature, filtering the slurry and drying to isolate the solids of Form B.

In another aspect, provided herein is a method of making the Form B polymorph of Compound I, the method comprising slurrying Compound I Form A in anhydrous ethanol at elevated temperature.

In another aspect, provided herein is a method of making the Form B polymorph of Compound I, the method comprising dissolving Compound I in ethanol and water and crystallization by adding water as antisolvent.

In another aspect, provided herein is a method of making the Form C polymorph of Compound I, the method comprising dissolving Compound I in a solvent mixture of THF and methanol, evaporating the solvent mixture, suspending the resulting solids in a solvent mixture of ethanol and water, and heating and then cooling the suspension.

In another aspect, provided herein is a method of making the Form C polymorph of Compound I, the method comprising slurrying Compound I Form A in ethanol followed by slurrying in THF, heating and then cooling the slurry, filtering the slurry and drying to isolate Form C.

In another aspect, provided herein is a method of making the Form C polymorph of Compound I, the method comprising drying Compound I Form F in an oven at about 40° C. for at least about 24 hours to produce Form C.

In another aspect, provided herein is a method of making the Form C polymorph of Compound I, the method comprising suspending Compound I Form F in a mixture of ethanol and water, and drying the solids to isolate Form C.

In another aspect, provided herein is a method of making the Form C polymorph of Compound I, the method comprising dissolving Compound I in a mixture of ethanol and water at elevated temperature, cooling the solution to about room temperature, and then adding water.

In another aspect, provided herein is a method of making the Form C polymorph of Compound I, the method comprising dissolving Compound I in THF, removing the THF by rotary evaporation, slurrying the resulting material in ethanol and heptane, and thermally cycling the resulting slurry.

In another aspect, provided herein is a method of making the Form D polymorph of Compound I, the method comprising heating Compound I Form A to about 250° C. and cooling to about 30° C.

In another aspect, provided herein is a method of making the Form E polymorph of Compound I, the method comprising slurrying Compound I Form A in anhydrous ethanol and thermally cycling the slurry.

In another aspect, provided herein is a method of making the Form E polymorph of Compound I, the method comprising slurrying Compound I Form A in anhydrous ethanol and stirring the resulting suspension, filtering the slurry and drying.

In another aspect, provided herein is a method of making the Form E polymorph of Compound I, the method comprising suspending Compound I Form F in ethanol.

In another aspect, provided herein is a method of making the Form E polymorph of Compound I, the method comprising suspending Compound I Form A in ethanol and shaking the resulting suspension, filtering the slurry and drying.

In another aspect, provided herein is a method of making the Form E polymorph of Compound I, the method comprising suspending Compound I Form A in ethanol, stirring the resulting suspension, decanting the mother liquor, drying the solids, adding additional ethanol and stirring the compound in the ethanol, filtering and drying to isolate the solids of Form E.

In another aspect, provided herein is a method of making the Form F polymorph of Compound I, the method comprising dissolving Compound I in a solvent mixture of THF and methanol, evaporating the solvent mixture, suspending the resulting solids in a mixture of isopropanol and water, heating the suspension and then cooling the solution.

In another aspect, provided herein is a method of making the Form F polymorph of Compound I, the method comprising dissolving Compound I in a solvent mixture of THF and methanol, evaporating the solvent mixture, suspending the resulting solids in a mixture of THF and water, heating the suspension and then cooling the solution, followed by solvent evaporation.

In another aspect, provided herein is a method of making the Form F polymorph of Compound I, the method comprising dissolving Compound I in THF, removing the THF by rotary evaporation, slurrying the resulting solids in isopropyl acetate, and thermally cycling the resulting slurry, followed by filtration of slurry and drying.

In another aspect, provided herein is a method of making the Form F polymorph of Compound I, the method comprising dissolving Compound I in a mixture of THF and water at elevated temperature, polish filtering the solution, partially removing the THF by distillation, cooling the slurry, adding 1-butanol to the slurry and further cooling the slurry.

In some embodiments of the various aspects of the methods of preparation, the crystalline form produced is substantially pure, i.e., substantially free of other forms of Compound I.

### IV. Pharmaceutical Compositions

In one aspect, provided herein is a pharmaceutical composition comprising a crystalline form disclosed herein (or combinations thereof) and a pharmaceutically acceptable excipient. In another aspect, provided herein is a method of preparing a pharmaceutical composition comprising providing a crystalline form disclosed herein and one or more pharmaceutically acceptable excipient(s) and forming a pharmaceutical composition from the crystalline form and the excipient(s).

In one aspect, the pharmaceutical compositions are useful for treating hypertrophic cardiomyopathy (HCM), heart failure with preserved ejection fraction (HFpEF) and other diastolic dysfunctions in humans and other subjects. The active agent is generally included in an amount sufficient to produce the desired effect upon myocardial contractility (e.g., to decrease the often supranormal systolic contractility in HCM) and to improve left ventricular relaxation in diastole. Such improved relaxation can alleviate symptoms in hypertrophic cardiomyopathy and other etiologies of diastolic dysfunction.

The pharmaceutical compositions for the administration of the compounds provided herein may conveniently be presented in unit dosage form and may be prepared by any of the methods known in the art of pharmacy and drug delivery. All methods include the step of bringing the active ingredient into association with a carrier containing one or more accessory ingredients. In general, the pharmaceutical compositions are prepared by uniformly and intimately bringing the crystal form of the active ingredient into association with a finely divided solid carrier, and then, if necessary, shaping the product into the desired formulation.

The pharmaceutical compositions containing the crystalline form may be in a form suitable for oral use, for example, as tablets, troches, lozenges, aqueous or oily suspensions, dispersible powders or granules, hard or soft capsules, a buccal patch, chewing gum, or chewable tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time-delay material such as glyceryl monostearate or glyceryl distearate may be employed. They may also be coated to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a liquid or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions provided herein may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a suspending medium. For this purpose any bland fixed oil may be employed including synthetic mono- or diglycerides. In addition, fatty acids such as oleic acid find use in the preparation of injectables.

The compounds provided herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds provided herein are employed.

The compounds of this invention may also be coupled to a carrier that is a suitable polymer for targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds provided herein may be coupled to a carrier that is a biodegradable polymer useful in achieving controlled release of a drug, such as polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

### V. Methods of Treating Cardiac Disorders

Provided herein is a method of treating hypertrophic cardiomyopathy (HCM). The method includes administering to a subject in need thereof an effective amount of a crystalline form as provided herein (or a pharmaceutical composition as provided herein). In some embodiments, the HCM is obstructive HCM. In some embodiments, the HCM is non-obstructive HCM.

Also provided herein is a method of treating heart failure with preserved ejection fraction (HFpEF). The method includes administering to a subject in need thereof an effective amount of a crystalline form as provided herein (or a pharmaceutical composition as provided herein).

Also provided herein is a method of treating diastolic dysfunction. The method includes administering to a subject in need thereof an effective amount of a crystalline form as provided herein (or a pharmaceutical composition as provided herein).

Also provided herein is a method of treating left ventricular hypertrophy (LVH). The method includes administering to a subject in need thereof an effective amount of a crystalline form as provided herein (or a pharmaceutical composition as provided herein).

The mutations that lead to HCM cause significant perturbations in myosin mechanics. These mutations exert their effects via distinct mechanisms depending on their locations in the myosin gene. The well-studied HCM mutations, R403Q and R453C, are located in different sections of the motor domain and cause distinct mechanistic perturbations that lead to the common outcome of increased force production. Without wishing to be bound by any particular theory, it is believed that the compounds provided herein can bind directly to the mutant sarcomeric proteins and correct for their aberrant function, either in cis (by affecting the same specific function) or in trans (by altering a complementary function). As such, they can provide therapeutic benefit for HCM patients by counteracting the hypercontractile and/or impaired relaxation associated with this disease.

Accordingly, provided herein is a method of treating hypertrophic cardiomyopathy (HCM) or a cardiac disorder having one or more pathophysiological features associated with HCM. The method includes administering to a subject in need thereof an effective amount of a compound provided herein, or a pharmaceutical composition thereof.

Diastolic dysfunction is present or an important feature of a series of diseases including, but not limited to, hypertrophic cardiomyopathy (HCM), heart failure with preserved ejection fraction (HFpEF)—including both disorders of active relaxation and disorders of chamber stiffness (diabetic HFpEF); dilated cardiomyopathy (DCM), ischemic cardiomyopathy, cardiac transplant allograft vasculopathy, restrictive cardiomyopathy—including inflammatory subgroups (e.g., Loefilers and EMF), infiltrative subgroups (e.g., amyloid, sarcoid and XRT), storage subgroups (e.g., hemochromatosis, Fabry and glycogen storage disease), idiopathic/inherited subgroups such as Trop I (beta myosin HC), Trop T (alpha cardiac actin) and desmin related (usually includes skeletal muscle), congenital heart disease subgroups (including pressure-overloaded RV, Tetrology of Fallot (diastolic dysfunction pre-op and early post-op, systolic dysfunction post-op) and pulmonic stenosis), and valvular heart disease (e.g., aortic stenosis—including elderly post AVR/TAVR and congenital forms).

Further determining factors for diagnosing diastolic dysfunction using echocardiography are described in J Am Soc Echocardiogr. 29(4):277-314 (2016), the contents of which are incorporated herein for all purposes.

Subjects in need of treatment for diastolic dysfunction include subjects from a patient population characterized by non-obstructive hypertrophic cardiomyopathy, or subjects characterized by heart failure with preserved ejection fraction (HFpEF). Subjects in need of treatment for diastolic dysfunction include subjects who exhibit left ventricle stiffness as measured by echocardiography or left ventricle stiffness as measured by cardiac magnetic resonance. In some embodiments, the subject in need thereof is from a patient population characterized by HFpEF.

Also provided herein is a method of treating a disease or disorder selected from the group consisting of diastolic heart failure (for example diastolic heart failure with preserved ejection fraction), ischemic heart disease, angina pectoris, and restrictive cardiomyopathy, comprising administering to a subject in need thereof an effective amount of a compound disclosed herein or a pharmaceutical composition thereof.

Also provided herein is a method of treating a disease or disorder characterized by left ventricular hypertrophy (for example due to volume or pressure overload), said disease or disorder selected from the group consisting of chronic mitral regurgitation, chronic aortic stenosis, and chronic systemic hypertension, optionally combined with therapies aimed at correcting or alleviating the primary cause of volume or pressure overload, including valve repair/replacement or effective antihypertensive therapy, comprising administering to a subject in need thereof an effective amount of a compound disclosed herein, or a pharmaceutical composition thereof.

Also provided herein is a method of treating hypertrophic cardiomyopathy (HCM), or a cardiac disorder (for example a cardiac disorder having a pathophysiological feature associated with HCM), comprising administering to a subject in need thereof an effective amount of a compound disclosed herein, or a pharmaceutical composition thereof, combined with therapies that retard the progression of heart failure by down-regulating neurohormonal stimulation of the heart and attempt to prevent cardiac remodeling (e.g., ACE inhibitors, angiotensin receptor blockers (ARBs), β-blockers, aldosterone receptor antagonists, or neural endopeptidase inhibitors); therapies that improve cardiac function by stimulating cardiac contractility (e.g., positive inotropic agents, such as the β-adrenergic agonist dobutamine or the phosphodiesterase inhibitor milrinone); and/or therapies that reduce cardiac preload (e.g., diuretics, such as furosemide) or afterload (vasodilators of any class, including but not limited to calcium channel blockers, phosphodiesterase inhibitors, endothelin receptor antagonists, renin inhibitors, or smooth muscle myosin modulators).

Also provided herein is a crystalline form as disclosed herein, or a pharmaceutical composition thereof, for use as a medicament.

Also provided herein is a crystalline form disclosed herein, or a pharmaceutical composition thereof, for use in the treatment of a disease or disorder as disclosed herein, e.g., hypertrophic cardiomyopathy, or other cardiac disorder.

Also provided herein is a use of a crystalline form as disclosed herein, or a pharmaceutical composition thereof, for the manufacture of a medicament for the treatment of a disease or disorder as disclosed herein, e.g., hypertrophic cardiomyopathy, or other cardiac disorder.

The compounds disclosed herein can alter the natural history of HCM and other diseases rather than merely palliating symptoms. The mechanisms conferring clinical benefit to HCM patients can extend to patients with other forms of heart disease sharing similar pathophysiology, with or without demonstrable genetic influence. For example, an effective treatment for HCM, by improving ventricular relaxation during diastole, can also be effective in a broader population characterized by diastolic dysfunction. The compounds can specifically target the root causes of the conditions or act upon other downstream pathways. Accordingly, the compounds can also confer benefit to patients suffering from diastolic heart failure with preserved ejection fraction, ischemic heart disease, angina pectoris, or restrictive cardiomyopathy. The compounds can also promote salutary ventricular remodeling of left ventricular hypertrophy due to volume or pressure overload; e.g., chronic mitral regurgitation, chronic aortic stenosis, or chronic systemic hypertension; in conjunction with therapies aimed at correcting or alleviating the primary cause of volume or pressure overload (valve repair/replacement, effective antihypertensive therapy). By reducing left ventricular filling pressure the compounds could reduce the risk of pulmonary edema and respiratory failure. Reducing or eliminating functional mitral regurgitation and/or lowering left atrial pressures may reduce the risk of paroxysmal or permanent atrial fibrillation, and with it reduce the attendant risk of arterial thromboembolic complications including but not limited to cerebral arterial embolic stroke. Reducing or eliminating either dynamic and/or static left ventricular outflow obstruction may reduce the likelihood of requiring septal reduction therapy, either surgical or percutaneous, with their attendant risks of short and long-term complications.

Depending on the disease to be treated and the subject's condition, the compounds provided herein may be administered by oral, parenteral (e.g., intramuscular, intraperitoneal, intravenous, ICV, intracisternal injection or infusion, subcutaneous injection, or implant), by implantation (e.g., as when the compound is coupled to a stent device), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In the treatment or prevention of conditions which require improved ventricular relaxation during diastole, an appropriate dosage level of Compound I will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. The pharmaceutical compositions preferably contain 1.0 to 1000 milligrams of Compound I, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams for the symptomatic adjustment of the dosage to the patient to be treated. Such compositions may be for oral administration, for example provided in the form of tablets or capsules.

The compounds, or pharmaceutical compositions thereof, may be administered on a regimen of 1 to 4 times per day, preferably once or twice per day.

It will be understood that the specific dose level and frequency of dosage for any particular patient may be varied and will depend upon a variety of factors including the activity of the specific compound, or pharmaceutically acceptable salt, employed, the metabolic stability and length of action of that compound or pharmaceutically acceptable salt thereof, the age, body weight, hereditary characteristics, general health, sex and diet of the subject, as well as the mode and time of administration, rate of excretion, drug combination, and the severity of the particular condition for the subject undergoing therapy.

Compounds and/or pharmaceutical compositions provided herein may be used in combination with other drugs that are used in the treatment, prevention, suppression or amelioration of the diseases or conditions for which compounds and compositions provided herein are useful. Such other drugs may be administered, by a route and in an amount commonly used therefor, contemporaneously or sequentially with a compound or composition provided herein. When a compound or composition provided herein is used contemporaneously with one or more other drugs, a pharmaceutical composition containing such other drugs in addition to the compound or composition provided herein is preferred. Accordingly, the pharmaceutical compositions provided herein include those that also contain one or more other active ingredients or therapeutic agents, in addition to a compound or composition provided herein. The weight ratio of the compound provided herein to the second active ingredient may be varied and will depend upon the effective dose of each ingredient. Generally, an effective dose of each will be used. Where a compound of the invention is administered in combination with another therapeutic agent, the other therapeutic agent can be administered simultaneously, separately or sequentially with the compound of the invention. The precise dosage regimen being commensurate with the properties of the therapeutic agent(s).

### VI. Examples

### Example 1: Characterization of Forms

### X-Ray Powder Diffraction (XRPD):

XRPD data of Forms B, E, and F were obtained using Bruker D8 Discover DaVinci with XYZ Stage. The IμS X-ray generator was operated at 50 kV and 1 mA with a Cu target (CuKα radiation). Incident beam optics included Montel mirrors with a 0.3 mm collimator. Photons were counted using an Eiger2 R 500K Detector in 2D, 2θ optimized mode. The sample-to-detector distance was 137.7 mm. Each sample was loaded into a glass capillary (1 mm diameter). Data were collected over a 2θ range of approximately 2-32° with an exposure time of 1000 s and an approximate step size of 0.01°, and at room temperature.

XRPD data of Forms C and D were obtained using a PANalytical X'pert Pro with a Cu target (CuKα radiation). The X-ray generator was operated at 40 kV and 40 mA with a Cu target (CuKα radiation) running in transmission mode. Each sample was lightly ground and loaded onto a multi-well plate with Kapton or Mylar polymer film. Data were collected over a 2θ range of approximately 3-35° with an approximate step size of 0.01°, and at room temperature.

### Differential Scanning Calorimetry (DSC):

The differential scanning calorimetry (DSC) experiments for Form B were performed using a TA Instruments—Discovery DSC 2500 with RCS90. Each sample was placed in a TZero aluminum DSC pan with a TZero lid, and the weight was accurately recorded. Data were collected between room temperature and a maximum temperature of approximately 350° C. at a heating rate of 10° C./min.

The differential scanning calorimetry (DSC) experiments for Forms C and E were performed using a Seiko DSC6200. Each sample was placed in a hermetically sealed aluminum DSC pan with a pierced lid, and the weight was accurately recorded. Data were collected between 20° C. and a maximum temperature of approximately 330° C. at a heating rate of 10° C./min.

Forms D and F can be analyzed by DSC by either of the above methods.

### Thermogravimetric Analysis (TGA):

The thermal gravimetric analysis (TGA) experiments for Form B were performed using a TA Instruments—Discovery TGA model 5500. The sample (˜10 mg) was placed in a previously cleaned and tarred platinum pan then loaded into the instrument furnace. The furnace was heated under nitrogen gas. Data were collected between room temperature and approximately 350° C. at a heating rate of 10° C./min.

Simultaneous thermogravimetric/differential thermal analysis (TG/DTA) experiments for Forms C, D, and E were performed using a Seiko TG/DTA 7200. Each sample (˜5 mg) was placed in an open aluminum pan then loaded into the instrument furnace. The furnace was heated under nitrogen gas. Data were collected between 20° C. and approximately 400° C. at a heating rate of 10° C./min.

Form F can be analyzed by TGA by either of the above methods.

### Vapor Sorption:

The vapor sorption experiments for Form B were performed using a TA Instrument VTI-SA+ Vapor Sorption Analyzer. Sample (˜10 mg) was placed in a previously cleaned and tarred platinum pan then loaded into the instrument chamber. Sample was dried at 60° C. until the loss rate of 0.005 wt %/min was obtained for 10 minutes. The sample was then held isothermally at 25° C. while the % RH of the system was stepped between 4, 5, 15, 25, 35, 45, 55, 65, 75, 85, 95, 85, 75, 65, 55, 45, 35, 25, 15, 5, 4% RH. Equilibration criteria was +/−0.01 wt % for 35 min with a maximum equilibration time of 600 min at each step.

Forms C, D, E, and F can be analyzed by vapor sorption by the above method. In particular, the procedure is well suited for anhydrate forms C, D, and E.

### Single Crystal X-Ray Analysis:

Single crystal X-ray data of Form B was collected using a Bruker D8-Venture diffractometer equipped with a Photon III detector and monochromatic Cu Kα radiation. The single crystals were held at 100K during data collection. Indexing and processing of the measured intensity data were carried out with the APEX3 program suite (Bruker AXS, Inc., 5465 East Cheryl Parkway, Madison, WI 53711 USA). The final unit cell parameters were determined using the full data set. The structures were solved by intrinsic phasing methods and refined by full-matrix least-squares approach using the SHELXTL software package (G. M. Sheldrick, SHELXTL v2018/3, Bruker AXS, Madison, WI USA). Structure refinements involved minimization of the function defined by Σw(|Fo|−|Fc|)2, where w is an appropriate weighting factor based on errors in the observed intensities, Fo is the structure factor based on measured reflections, and Fc is the structure factor based on calculated reflections. Agreement between the refined crystal structure model and the experimental X-ray diffraction data is assessed by using the residual factors R=Σ∥Fo|−|Fc∥/Σ|Fo| and wR=[Σw(|Fo|−|Fo|]½. Difference Fourier maps were examined at all stages of refinement. All non-hydrogen atoms were refined with anisotropic thermal displacement parameters. Hydrogen atoms on carbon atoms were introduced using idealized geometry with isotropic temperature factors and included in structure factor calculations with fixed parameters. Hydrogen atoms on heteroatoms were located from residual electron density and freely refined.

Single crystal X-ray data of Form E was collected using a Bruker D8-Venture diffractometer equipped with a Photon II detector and monochromatic Mo Kα radiation. The single crystals were held at 302K during data collection. Indexing and processing of the measured intensity data were carried out with the APEX3 program suite (Bruker AXS, Inc., 5465 East Cheryl Parkway, Madison, WI 53711 USA). The final unit cell parameters were determined using the full data set. The structures were solved by intrinsic phasing methods and refined by full-matrix least-squares approach using the SHELXTL software package (G. M. Sheldrick, SHELXTL v2016/6, Bruker AXS, Madison, WI USA.). Structure refinements involved minimization of the function defined by Σw(|Fo|−|Fc|)2, where w is an appropriate weighting factor based on errors in the observed intensities, Fo is the structure factor based on measured reflections, and Fc is the structure factor based on calculated reflections. Agreement between the refined crystal structure model and the experimental X-ray diffraction data is assessed by using the residual factors R=Σ∥Fo|−|Fc|/Σ|Fo| and wR=[Σw(|Fo|−|Fc|]½. Difference Fourier maps were examined at all stages of refinement. All non-hydrogen atoms were refined with anisotropic thermal displacement parameters. Hydrogen atoms on all atoms were introduced using idealized geometry with isotropic temperature factors and included in structure factor calculations with fixed parameters.

### Example 2. Preparation of Form B

### Example 2A

Compound I was dissolved in tetrahydrofuran (THF) and solvent was removed by rotary evaporation. 400 mg of the resulting solids was slurried in 12 mL of ethanol. The slurry was thermally cycled between ambient and 40° C. in 4 hour cycles with agitation for 96 hours. The slurry was filtered and dried at ambient for 1 hour. Solids consisted of Form B.

### Example 2B

2.3 g of Form A was slurried in 2θ mL ethanol. The slurry was split between 2 vials and an additional 10 mL ethanol was added to each vial. The slurries were thermally cycled between ambient and 40° C. in 4 hour cycles with agitation. The samples were filtered together and dried at 40° C. under vacuum for 90 minutes. 2θ mL of ethanol was added to the material and the slurry was stirred at 60° C. for 72 hours. The slurry was filtered, dried at 40° C. for 2 hours. Solids consisted of Form B.

### Example 2C

3 g of Form A was slurried in 90 mL anhydrous ethanol at 60° C. for 24 hours. Solids consisted of Form B. The conversion of Form A to Form B demonstrated the superior thermodynamic stability of Form B compared to Form A.

### Example 2D

A direct crystallization (with seeds) process was performed to produce Form B. Ethanol (504.30 kg), water (197.70 kg) and Compound I Form F (31.76 kg) were charged to a 2000 L reactor. The charging port was rinsed with ethanol (141.15 kg). The reaction mass was warmed to 75±5° C. The reaction mass was stirred at 75±5° C. for 0.5 hours (clear solution). The reaction mass was cooled to 60±2° C. The seed of Form B in ethanol suspension (0.5% w/w in 175.5 g in ethanol) was charged to the reaction mass at 60±2° C. The slurry mass was stirred at 60±2° C. for 1 hour. The slurry mass was cooled to 58±4° C. The slurry mass was stirred at 58±4° C. for 2 hours. Water (64.05 kg) was charged to the slurry mass at 58±4° C. over 1 hour. The slurry mass was stirred at 58±4° C. for 3 hours. Water (51.00 kg) was charged to the slurry mass at 58±4° C. over 1 hour. Water (160.50 kg) was continued to be charged to the slurry mass at 58±4° C. over 2.5 hours. Water (319.70 kg) was continued to be charged to the slurry mass at 58±4° C. over 3 hours. The slurry mass was stirred at 58±4° C. for 3 hours. The slurry mass was cooled to 10±5° C. over 5 hours. The slurry mass was stirred at 10±5° C. for 4 hours. The slurry mass was filtered by centrifugation. The filter cake was rinsed with ethanol-water mixture (1:1, 51.00 kg+63.95 kg). The filter cake was dried at 50±10° C. for 10 hours. 28.95 kg of Compound I Form B was obtained.

### Example 3. Preparation of Form C

### Example 3A

8 mg of Compound I was dissolved in 1 mL of 80:20 THF:MeOH. Solvent was evaporated at 40° C. under nitrogen gas flow. The resulting solids were suspended in 800 uL 50:50 Ethanol:water with magnetic stirring at room temperature. Suspension was heated to 80° C., which resulted in dissolution of solids. The solution was cooled to 10° C. with cooling rate of 0.1° C./min, resulting in a suspension. The solids consisted of Form C.

### Example 3B

500 mg of Form A was slurried in 2θ mL of ethanol at ambient for 16 h. The slurry was filtered and re-slurried in 10 mL of tetrahydrofuran, heated to 40° C. for 1 hour and cooled to ambient. The slurry was filtered and dried at 40° C. for 16 hours. Solids consisted of Form C.

### Example 3C

500 mg of Form F material was dried in an oven at 40° C. under vacuum for 24 hours.

Solids consisted of Form C.

### Example 3D

100 mg of Form F was suspended in ethanol (0.25 mL) and water (3.75 mL) at room temperature overnight. The solids were dried at 50° C. for 2 days. The solids consisted of Form C.

### Example 3E

200 mg of Compound I was dissolved in 5 mL ethanol and 1.24 mL water at 80° C. The solution was cooled to room temperature. 3.72 mL of water was added in less than a minute, which immediately resulted in slurry. The solids consisted of Form C.

### Example 3F

Compound I was dissolved in tetrahydrofuran (THF) and solvent was removed by rotary evaporation. 50 mg of the resulting material was slurried in 50:50 ethanol:heptane before being thermally cycled between ambient and 40° C. in 4-hour cycles for 24 hours. Solids consisted of Form C.

### Example 4. Preparation of Form D

Form A was heated to 250° C. and immediately cooled down to 30° C. The solids consisted of Form D.

### Example 5. Preparation of Form E

### Example 5A

6 g of Form A was slurried in 180 mL anhydrous ethanol. The slurry was thermally cycled between 20° C. and 40° C. in 4 hour cycles with agitation for 96 hours. Solids consisted of Form E.

### Example 5B

3 g of Form A was slurried in 90 mL anhydrous ethanol. The suspension was stirred at 25° C. for 24 hours. Solid was collected by filtration and dried at 35° C. overnight. Solids consisted of Form E.

### Example 5C

100 mg of Form F was suspended in 3 mL ethanol at room temperature overnight. The resulting solids were Form E.

### Example 5D

200 mg of Form A was suspended in 3 mL of ethanol and shaken at ambient temperature for 72 hours. The material was then dried at ambient for 21 hours. Solids consisted of Form E.

### Example 5E

500 mg batch of Form A was suspended in 7.5 mL of ethanol. The slurry was stirred at ambient temperature for 2θ hours. The mother liquor was decanted, and the solids were dried in an oven at 40° C. under vacuum for 24 hours. 50 mg of material was retained, and 7.5 mL of ethanol was added to the remaining material. The slurry was stirred at ambient for 96 hours. Material was then dried in an oven at 40° C. under vacuum for 24 hours. Solids consisted of Form E.

### Example 6. Preparation of Form F

### Example 6A

8 mg of Compound I was dissolved in 1 mL of 80:20 THF:MeOH. Solvent was evaporated at 40° C. under N2 gas flow. The resulting solids were suspended in 400 uL 50:50 isopropanol (IPA):water with magnetic stirring. Suspension was heated to 80° C., which resulted in dissolution of solids. The solution was cooled to 10° C. with cooling rate of 0.1° C./min, resulting in a suspension. The solids consisted of Form F.

### Example 6B

8 mg of Compound I was dissolved in 1 mL of 80:20 THF:MeOH. Solvent was evaporated at 40° C. under N2 gas flow. The resulting solids were suspended in 400 uL 19:174 water:THF with magnetic stirring. Suspension was heated to 60° C., which resulted in dissolution of solids. The solution was cooled to 10° C. and solvent was evaporated under nitrogen. The solids consisted of Form F.

### Example 6C

Compound I was dissolved in THF and solvent was removed by rotary evaporation. 400 mg of the resulting solids was slurried in 12 mL of isopropyl acetate. The slurry was then thermally cycled between ambient and 40° C. in 4 hour cycles with agitation for 24 hours. The slurry was then filtered and dried at ambient for 1 hour. The solids consisted of Form F.

### Example 6D

70 g of Compound I was dissolved in 890 mL of 90:10 THF:water at 65° C. under agitation in a reactor. Solution was removed from the reactor and polish filtered. Solution was transferred back to the reactor. Slurry was observed at 45° C. THF was removed by distillation at 85° C. until the volume of the slurry reached 300 mL. Slurry was cooled to 60° C. and 555 mL of 1-butanol was added. The batch was cooled to 20° C. and aged overnight. The solids consisted of Form F.

### Example 7. Preparation of Form a

U.S. Patent Appl. Publ. No. 2020/0165247 A1, which is incorporated herein by reference in its entirety, discloses Form A of Compound I, referred to therein as the Form 1 polymorph. The X-ray powder diffraction pattern of Form A (i.e., Form 1 polymorph) is provided as FIG. 1 of the present disclosure. Form A can also be prepared according to the following method: Methanol (MeOH) (154.70 kg) was charged to a 300 L reactor under N₂. The solution was cooled to −10±5° C. Compound I Form F (7.8 kg) was charged to the reactor. The feeding port was rinsed with MeOH (12.0 kg). The slurry mass was stirred for 2 hours at −10±5° C. A sample was taken for XRPD. The pattern of Form F completely disappeared. The slurry mass was filtered by centrifugation. The filter cake was rinsed with MeOH (12.1 kg) and 3.69 kg of the wet material was obtained. The cloudy mother liquor was filtered by vacuum filtration. The filter cake was rinsed with MeOH (24.70 kg*2) and 7.77 kg of wet material was obtained. The material was dried by gradual increase of oven temperature to 65±10° C. over 38 hours, and finally was dried at 65±10° C. for 24 hours until the MeOH content was less than 0.3% by 1H NMR. 6.0 kg of Compound I material was obtained, which was characterized as Form A.

### Example 8. Stability Studies

Four anhydrate forms, Forms A, B, C and E, were evaluated for relative stability. Form B was identified as a pharmaceutically useful form of particular interest, as evidenced by its thermodynamic stability in competitive slurry testing. Further testing of Form B demonstrated that Form B remained unchanged after shaking for 24 hours in certain bio-relevant media and Form B remained stable when subjected to thermal and humidity treatments.

### Competitive Slurry

Competitive slurry experiments between Form B and Form E in ethanol showed that Form B is more stable compared to Form E at 22.5° C. and 60° C. Competitive slurry experiments between Form B and Form C in ethanol showed that Form B is more stable compared to Form C at 10° C. and 60° C. Competitive slurry experiments between Form B and Form A in ethanol showed that Form B is more stable compared to Form A at 10° C. and 60° C. The results have shown that Form B is the thermodynamically most stable anhydrous form compared to Forms A, C and E at temperature ranges covered in these experiments.

### Solid State Stability in Bio-relevant Media

Form B was added to bio-relevant media (pH 1 HCl 0.1 M; pH 3.05 citrate 50 mM; pH 7.4 phosphate 50 mM; Fassif pH 6.5; Fessif pH 5.0) and solid state stability in the media was determined. Procedures are described in more detail as follows:

### Preparation of Bio-Relevant Media

pH 1 HCl 0.1M: 8.33 mL HCl was diluted to 100 mL with water. pH 1.02

pH 3.05 citrate 50 mM: 82 mL of 0.1 M citric acid and 18 mL of 0.1 M sodium citrate were mixed together. pH=3.07.

pH 7.4 phosphate 50 mM: 50 mL of 0.2 M KH₂PO₄ and 39.1 mL of 0.2 M NaOH were dissolved and diluted to 200 mL with water. pH 7.39.

Fassif pH 6.5: 23.4 mg of NaOH, 224.7 mg of NaH₂PO₄·2H₂O and 304.3 mg of NaCl were dissolved and diluted to 50 mL with water. 33.6 mg of SIF powder was dissolved in 15 mL of FaSSIF buffer solution. The solution was kept stirring and equilibrated at ambient condition with light protection for 2 hours. pH=6.37.

Fessif pH 5.0: 202.6 mg of NaOH, 432.7 mg of acetic acid, and 591.3 mg of NaCl were dissolved and diluted to 50 mL with water. pH of the solution was 4.99. 168 mg of SIF powder was dissolved in 15 mL of FeSSIF buffer solution. The solution was kept stirring and equilibrated at ambient condition with light protection for 2 hours. pH=4.94.

### Preparation of Test Solutions

30-50 mg of Form B was weighed into 12 mL sample vials and then 3 mL of test media was added respectively. The suspensions were kept shaking at 200 rpm under 37° C. for 24 hours. After 24 hours, the suspensions were filtered and the filter cakes were characterized by XRPD.

The results showed that Form B was stable in bio-relevant media under the conditions tested and remained as Form B by XRPD after 24 hours in the media.

### Stability Study of Form B

The stability study of Form B (before milling) was carried out at 80° C. for two weeks and at 40° C./75% RH for one month. Purity was determined by HPLC and stability was determined by XRPD. The purity (99.9%, by HPLC) and crystal form (by XRPD) remained unchanged after test, suggesting that Form B is stable under those conditions. The stability of milled Form B was carried out as well under the same conditions. Milled Form B did not show any degradation after being placed at 40° C./75% RH for four weeks. A purity decrease of 0.8% was detected for milled Form B after being treated at 80° C. for four weeks. Based on XRPD, Form B remains unchanged after thermal and humidity treatments.

### Other Embodiments

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. Form B polymorph of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione.

2. The polymorph of claim 1, characterized by an X-ray powder diffraction pattern, obtained by irradiation with Cu-Kα at room temperature, comprising at least three peaks selected from the group consisting of 5.5, 7.1, 9.3, 16.5, and 19.0 °2θ±0.2 °2θ.

3-6. (canceled)

7. The polymorph of claim 1, characterized by an X-ray powder diffraction pattern, obtained by irradiation with Cu-Kα at room temperature, comprising peaks at 5.5, 7.1, 9.3, 16.5, 19.0, and 22.3 °2θ±0.2 °2θ.

8. The polymorph of claim 1, characterized by an X-ray powder diffraction pattern, obtained by irradiation with Cu-Kα at room temperature, comprising a peak at 5.5 °2θ±0.2 °2θ and at least four peaks selected from the group consisting of 7.1, 8.7, 9.3, 13.8, 16.2, 16.5, 17.2, 19.0, and 22.3 °2θ±0.2 °2θ.

9-13. (canceled)

14. The polymorph of claim 1, characterized by an X-ray powder diffraction pattern, obtained by irradiation with Cu-Kα at room temperature, substantially as shown in FIG. 2.

15. The polymorph of claim 7, characterized by a DSC thermogram comprising an endotherm onset at about 307° C.

16. (canceled)

17. The polymorph of claim 15, characterized by a DSC thermogram substantially as shown in FIG. 3.

18-20. (canceled)

21. The polymorph of claim 7, characterized by a triclinic crystal system with a P1 space group.

22. (canceled)

23. (canceled)

24. The polymorph of claim 21, characterized by unit cell dimensions, at a temperature of 100 Kelvin, of a=6.74±0.10 Å, b=12.74±0.10 Å, c=15.99±0.10 Å, α=83.9±1.0°, β=80.0±1.0°, and γ=75.1±1.0°.

25-29. (canceled)

30. Form C polymorph of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione.

31. The polymorph of claim 30, characterized by an X-ray powder diffraction pattern, obtained by irradiation with Cu-Kα at room temperature, comprising at least three peaks selected from the group consisting of 7.5, 13.8, 16.4, 17.4, 20.1, and 27.7 °2θ±0.2 °2θ.

32-48. (canceled)

49. Form D polymorph of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione.

50. The polymorph of claim 49, characterized by an X-ray powder diffraction pattern, obtained by irradiation with Cu-Kα at room temperature, comprising at least three peaks selected from the group consisting of 11.1, 15.0, 18.3, 19.8, 20.2, 22.5, and 25.9 °2θ±0.2 °2θ.

51-64. (canceled)

65. Form E polymorph of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione.

66. The polymorph of claim 65, characterized by an X-ray powder diffraction pattern, obtained by irradiation with Cu-Kα at room temperature, comprising at least three peaks selected from the group consisting of 4.1, 8.6, 16.5, 17.7, and 23.2 °2θ±0.2 °2θ.

67-91. (canceled)

92. Form F polymorph of (6S,7S)-6-fluoro-7-(2-fluoro-5-methylphenyl)-3-(tetrahydro-2H-pyran-4-yl)-5,6,7,8-tetrahydropyrido[2,3-d]pyrimidine-2,4(1H,3H)-dione.

93. The polymorph of claim 92, characterized by an X-ray powder diffraction pattern, obtained by irradiation with Cu-Kα at room temperature, comprising at least three peaks selected from the group consisting of 6.8, 9.0, 13.7, 14.2, and 20.3 °2θ±0.2 °2θ.

94-108. (canceled)

109. A pharmaceutical composition comprising a polymorph of Form B according to claim 1 and a pharmaceutically acceptable excipient.

110. (canceled)

111. A method of treating a cardiac disease or disorder comprising administering to a subject in need thereof an effective amount of a polymorph of claim 1.

112-116. (canceled)

117. A method of preparing a pharmaceutical composition comprising providing a polymorph of claim 1 and one or more pharmaceutically acceptable excipients and forming a pharmaceutical composition from the polymorph and the one or more pharmaceutically acceptable excipients.
